(19) United States
(12) Patent Application Publication
(10) Pub. No.: US 2004/0101506 A1
(43) Pub. Date: May 27, 2004

(54) Composition for the prevention and treatment of inflammation of the ear

(76) Inventor: Charles A. Fust, Englewood, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/303,625

(22) Filed: Nov. 25, 2002

(51) Int. Cl. A61K 7/075; A61K 35/78

(52) U.S. Cl. 424/70.31; 424/74; 424/744

(57) ABSTRACT

A composition is disclosed for cleansing the ear canal, where the composition includes a carrier agent for ingredients and an antiseptic/antibacterial and/or anti-fungal agent. The disclosure relates to a composition for cleansing the ear canal, as well as preventing and treating infections of the ear. The composition may include a saline solution as a moisturizing base component, a buffering agent, a preservative, an antiseptic and/or anti-fungal agent, a counter-irritant, an alcohol, and/or an aloe concentrate. The disclosure is also related to the use of such composition to provide many unexpected benefits of a clean and healthy ear canal that may prevent the need for antibiotic and/or steroid treatment for ear infections.

### FIELD OF THE INVENTION

This invention relates to a composition that is applied to ears, and more particularly relates to a composition that helps prevent and treat inflammation of the ear.

### BACKGROUND OF THE INVENTION

Otitis externa, also known as swimmer's ear, is an inflammation of the skin of the canal between the eardrum and the external opening of the ear (the external auditory meatus). Otitis externa is commonly the result of infection with one or more of the following exemplary organisms: Acremonium; Alternata; Arachniotus citrinus, Arthrographis kalrae; Aspergillus fumigalus and niger; Aureobasidium; Beauveria; Bipolaris, Candida albicans, tropicalis, and pseudoiropicalis; Chaetomium, Chryosporium; Cladosporium herbarum; Cryptococcus, Curvularia, Epicoccum; Exophiala jeanselmei; Fusarium solani; Geotrichum, Gliomastix, Nigrospora, Mucor, Oldiodendron; Paecilomyces; Penicilliumr funiculosum; Phoma; Pithomyces; Pseudallescheria boydii; Pseudomonas; Rhinocladiella, Rhizopus; Rhodotorupa; Saccharomyces cerevisiae; Sagrahamala; Scolecobasidium; Scopulariopsis, Staphylococcus, Streptococcus; Trichoderma; Trichophyton; Trichosporon beigelli, and Ustilago.

The pain associated with bacterial otitis externa can be particularly excruciating. The pain is made worse by pressure on the outer ear. Some hearing loss usually occurs, as well as other ear-related symptoms such as tinnitus and vertigo.

This problem can be associated with the reduction in the thin coat of earwax due to over-aggressive cleansing of the ears with swabs and other methods such as ear canal irrigation. The skin on the outer part of the ear canal has special glands that produce earwax. The purpose of this natural wax is to repel water and to trap dust and sand particles. Additionally, earwax is slightly acidic, and this acidity tends to discourage growth of bacteria and fungi. Thus, earwax is the body's natural barrier against bacteria and fungi. When the wax barrier is removed, the body's defense against infection is compromised. High humidity and dampness in the ear canal create an environment in which the bacteria and fungi can thrive, and ultimately cause infection.

Conventional treatment for otitis externa depends upon the stage and severity of the disease, and usually is antibiotic in nature. Additional treatment may include corticosteroids, such as hydrocortisone, which serve to reduce inflammation. There is certain risk associated with conventional treatment, such as antibiotic immunity, or damaging side effects of steroid use such as increased appetite, indigestion, nervousness or restlessness, dizziness or lightheadedness, flushing of face or cheeks, sensation of spinning, etc.

In extreme cases of otitis externa, individuals may scratch at the source of infection, further irritating the skin and tissue in the ear canal, and in extreme cases, causing lesions to form. Common medications used to treat otitis externa utilize acetic acid as an active ingredient. While these ear solutions are highly effective in killing the germs which cause the infection (e.g., Staphylococcus) that cause the inflammation of the ear canal, applying such a low pH solution to the tender or inflamed areas can have a burning effect. The burning effect can lead to the patient further aggravating the problem by scratching at the area even more. This form of treatment is impractical in many applications, particularly in pediatrics and institutional environments such as nursing homes.

While the medical community has a wide variety of protocols available for the symptomatic treatment of otitis extema and other bacterial and/or fungi related diseases, it appears that little or no attention has been given to prevention of the disease. Traditionally, patients seek medical assistance after the infection has occurred. Seeking medical attention after the onset of infection may be complicated by an immune system response to the fungus and bacteria, resulting in inflammation of the ear canal or outer ear (externa). Solutions formulated for the removal of earwax (e.g., carbamide peroxide) and drying agents (e g., isopropyl alcohol) have not proven effective for the prevention or treatment of otitis.

### SUMMARY OF THE INVENTION

Embodiments of the present invention provide a composition and method for cleansing the ear canal and preventing and treating ear infections and inflammations. Briefly described, one embodiment of the composition, among others, includes a carrier agent; an antiseptic and/or anti-fungal agent; a buffering system; and a drying agent, wherein the drying agent helps prevent excess moisture in the ears and prevents an environment from forming in the ear canal that contributes to the growth of fungi, germs and bacteria. The antiseptic and/or anti-fungal agent may be present in the composition in an amount from approximately 0.001% to 10% by weight of the composition. Additionally, the composition may include aloe as a surfactant and/or soothing agent.

Embodiments of the present invention can also be viewed as providing methods for cleansing the ear canal, preventing ear infections, and/or treating ear infections. In this regard, one embodiment of such a method, among others, can be broadly summarized by the following steps: providing the composition described above, and washing the ear canal with the composition. The ear canal may be washed with the composition by delivering the composition with a measured dosage, pump atomizer spray dispenser.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### DETAILED DESCRIPTION

The present invention is a liquid composition that is dispensed into the ears. It is of particular utility for use by persons who suffer with chronic, acute otitis extema; however, the invention provides a routine method of treating the ears against colonization of fungi, germs and/or bacteria, thus rendering the composition of the present invention beneficial to all users.

The present invention includes introducing a composition into the ears containing an antiseptic and/or anti-fungal solution. For the purposes of this document, “antiseptic” means pertaining to or effecting the destruction of microorganisms that produce local or generalized invasion of the body by pathogenic microorganisms or their toxins. “Anti-fungal” means agents destructive to fungi, suppressing their growth and reproduction, and effective against fungal infections.

One of the uses of the composition is the elimination of fungi and/or bacteria known to cause acute otitis externa. The composition is designed as a preventative measure, as well as a treatment measure, and does not contain any habit-forming ingredients that would have an adverse physiological effect. Additionally, the composition should not react adversely to other medications the user may be taking.

The present invention also includes providing a composition that may be conveniently introduced with an atomizer-type sprayer, or other suitable delivery methods capable of saturating the inner ear canal. The methods of the present invention cause no discomfort to the user.

The composition includes a carrier agent, which may be water or a mildly diluted saline solution. The saline solution is preferred and provides a mild antibacterial effect as well as a mild drying effect. The concentration may vary from approximately 50% saline to approximately 75% saline; however, a solution of approximately 65% saline, buffered to be neutral or slightly acidic, e.g., approximately 6.5 to 7.0, has been found to be effective and non-objectionable for a user. Preferably, the carrier agent has a viscosity that optimizes the properties of any surfactant(s) or other ingredients added to the carrier agent.

Additionally, a small quantity of alcohol may be used in the composition to provide an antibacterial and drying effect. The alcohol is not necessary, but may also facilitate mixing of the other components. Additionally, a surfactant and/or agent may be added to the composition to improve viscosity, such as propylene glycol, aloe, or glycerin, for example.

A further component that may be added is a preserving agent such as benzalkonium chloride, which is used to extend shelf life and which can also be used as an anti-fungal agent. The use of the preserving agent or agents depends on whether or not some or any of the other components are stable during storage. In addition, a solubility enhancing ingredient may be included in the solution of the present invention, including for example, but not limited to, polysorbate and/or glycerin. An anhydrous glycerin base ingredient may thicken the composition, and further improve viscosity.

Zinc ions are powerful and natural antirhinoviral agents, immune system aids, interferon inducers, cell plasma/membrane pore closing agents, anti-inflammatory agents, antioxidants, protease inhibitors, and strong drying agents. The composition of the present invention may include also a compound or composition with zinc ion availability (ZIA) of 100, zinc acetate, or zinc chloride, thereby providing further healing and soothing properties to the composition. Mint extract oils can also be used in the composition of the present invention as a healing agent. Mint extract oils can soothe and moisturize tissue that may be abraded or inflamed in the ear canal thus aiding in the healing process.

Also included in the composition is an antiseptic and/or anti-microbial agent. Various embodiments of the composition of the invention exist in which various antiseptic and/or anti-microbial agents are used. In the preferred embodiment of the composition, the antiseptic agent used is cetylpyridinium chloride (CPC), which can also be used as an anti-fungal agent. Other antiseptic and/or anti-microbial agents include, but are not limited to, chlorhexidine digluconate, hexetidine, sanguinanine, triclosan, and benzalkonium chloride. One of ordinary skill in the art can envision that other similar antiseptic and/or anti-microbial agents may be substituted for those listed.

The present invention may also include other ingredients for healing and soothing, such as for example, but not limited to, aloe or aloe extract. Other possible ingredients include one or more of the following: an aid to the immune system, an interferon inducer, an anti-inflammatory, an antioxidant and a drying agent.

With respect to acute otitis extema, a forensic laboratory protocol has been developed to measure the mini-kill time test against exemplary organisms found in high concentrations of persons exhibiting the symptoms of acute otitis externa. The organisms tested include: Alternaria alternata, Aspergillus niger, Candida albicans, Cladosporium herbarum, Penicillium funiculosum and Fusarium solani, and their sub-species.

The procedure involves testing concentrations of a composition of the present invention in a liquid matrix for a pre-determined exposure time to demonstrate total kill of all organisms in the presence of the solution. This study supports the efficacy of the ingredients contained in the composition of the present invention, the results of which are conclusive and favorable. The results prove that routine, hygienic cleansing of the ear canal using the composition containing isotonic saline and antiseptic and/or anti-fungal ingredient(s) agent will result in cleaner, healthier, fungi- and/or bacteria-free ear canals. Users have reported relief of many types of symptoms, likely due to their routine programs of hygienic cleansing of the ear canals with this antiseptic and/or anti-fungal solution.

It should be noted that the composition of the present invention is not formulated to dissolve or remove all earwax from the ear canal as other earwax removal systems of the prior art. Thus, the composition may be used in the ears, and leave behind an appropriate amount of earwax that repels water and helps maintain healthy ears.

The following Table 1 provides a representative example of a composition of the invention, in weight/weight (w/w) volumes. Table 2 lists beneficial properties associated with the exemplary ingredients of Table 1.

TABLE 1
EXEMPLARY COMPOSITION

| Ingredient | Amount (w/w volume) |
|---|---|
| Sodium Chloride | 0.65% |
| Dibasic and Monobasic | 0.73 and 0.72 (Upper and Lower) |
| Sodium Phosphate | |
| Propylene Glycol | 3.00% |
| Glycerin | 0.045% |
| Cetylpyridinium Chloride | 0.01% |
| Aloe Concentrate | 2.00% |
| Zinc Acetate | 0.26% |
| Benzalkonium Chloride | 0.005% |
| Purified Water | Balance of Solution equaling 100% |

TABLE 2
PROPERTIES OF EXEMPLARY INGREDIENTS OF TABLE 1

| | |
|---|---|
| Ingredient | Amount |
| Sodium Chloride | Base Ingredient/Osmotic Agent |
| Dibasic and Monobasic | Buffering Agents, Upper and Lower pH Values |
| Sodium Phosphate | |
| Propylene Glycol | Surfactant/Drying Agent |
| Glycerin | Surfactant/Viscosity Improver |
| Cetylpyridinium Chloride | Antiseptic/Anti-bacterial |
| Aloe Concentrate | Surfactant/Anti-inflammatory |
| Zinc Acetate | Antioxidant/Drying Agent |
| Benzalkonium Chloride | Preservative/Anti-fungal |
| Purified Water | Solvent Vehicle |

The example of the composition given is representative of the preferred embodiment; however, the composition should not be limited to this example in terms of both ranges of the ingredients and administration of the composition. Many of the ingredients listed in Table 1 are optional. The formula in Table 1 of an exemplary composition has undergone testing and has proved effective for its stated purpose. The composition was dispensed utilizing an atomizer, pump sprayer specialized for ear applications and was successful in eliminating the fungi, germs and/or bacteria described hereinbefore.

Based on other related tests and formulations, many of the ingredients can be substituted with other similar ingredients, both in combinations and percentages, all resulting in similar conclusions. The base ingredients of the composition, preservative(s) and alcohol are, one embodiment, in percentage ratios that remain relatively constant. The solution of the invention is prepared according to known techniques and utilizing known excipients, for example, as described in “Remington's: The Science and Practice of Pharmacy,” Easton, Pa.: Mack Publishing Company; 1995 (“Remington's”). It should be noted that the composition of the invention is not limited to the exact ingredients stated herein. Substitute ingredients are widely available for most of the components having the properties described hereinabove, and are set forth in Remington's, for example.

The first set of ingredients of the composition is the antiseptic, anti-infective and/or anti-fungal agents. The elimination of certain types of fungi, germs and/or bacteria, as described in this document, which are known to cause acute otitis extema, will result in health improvement among users.

The second set of ingredients of the composition is the drying agents, which aid in the elimination of excess moisture, and thus, the elimination of the environment in which fungi, germs and/or bacteria are known to thrive.

The third set of optional ingredients of the composition includes the antirhinoviral agents, immune system aids, interferon inducers, cell plasma/membrane pore closers, anti-inflammatory agents, antioxidants, and protease inhibitors. Aspects of the composition, which can be varied, are the solution ratios of ingredients and surfactant properties, so as to provide properly balanced viscosity, solubility and surface dwell time, all relative to the application and user comfort. An additional consideration in the formula of the composition of the invention is the shelf life stability and preserved nature of the composition. Another aspect of the invention is the delivery method used for the composition, which has been found to be effective and user-friendly. Dispensing a proper amount in a convenient manner further increases the benefits of the composition.

The invention also includes a system and method, where the method includes the steps of: providing the composition of the invention detailed hereinbefore, and washing the ear canal with the composition. The composition may include the following: a carrier agent; an antiseptic and/or anti-fungal agent; a buffering system, wherein the buffering system may include, for example, at least one of dibasic sodium phosphate and monobasic sodium phosphate; and a drying agent, wherein the drying agent helps prevent excess moisture in the ears and prevents an environment from forming in the ear canal that contributes to the growth of fungi, germs and bacteria. Preferably, the antiseptic and/or anti-fungal agent is provided in an amount sufficient to prevent growth of bacteria and/or fungi. More preferably, the antiseptic and/or anti-fungal agent is present from approximately 0.001 percent to approximately 10 percent by weight of the composition. More preferably, the antiseptic and/or anti-fungal agent is present in an amount greater than 1 percent by weight of the carrier. It has been found that an amount of antiseptic greater than 1% by weight of the carrier effectively treats ear inflammations and may help prevent and/or treat ear infections. In particular, an amount of greater than 6% by weight anti-fungal agent is particularly effective for preventing and treating fungal-based infections. Thus, the method of cleansing the ear canal has a benefit of preventing ear infections and/or inflammations.

The method of cleansing the ear canal provides the additional benefit of soothing an ear canal that is already infected and reducing inflammation in an infected ear canal. The composition provided in the method preferably has a balanced viscosity range and surface dwell time, thereby rendering it user friendly. Additionally, the composition provided in the method preferably may include a preservative that increases the shelf life of the composition, thus increasing the length of time during which the composition is useful.

The system for dispensing or delivering the composition for washing the ear canal can vary. Delivery of the composition may be accomplished by, for example, but not limited to, spray bottles, droppers, and/or atomizer type squeeze or pump bottles. A measured dosage, pump atomizer spray dispenser has proven to be effective and is the preferred system for dispensing the composition. No user discomfort was experienced in the testing of the composition, when delivered in this manner.

The example of the composition given is representative of a preferred embodiment; however, the ranges of the composition should not be limited to this example in terms of ingredients and administration. Thus, while embodiments, and various modifications thereof, of the composition for cleansing and treating ear canals have been described in detail herein, various additional changes and modifications may be made without departing from the spirit or scope of the present invention.

Therefore, having thus described the invention, at least the following is claimed:

1. A composition for washing an ear canal comprising;
a carrier agent;
an antiseptic agent present in an amount sufficient to treat infection of the ear;
a buffering system, wherein the buffering system buffers the pH to be acidulous; and
a drying agent, wherein the drying agent helps prevent excess moisture in the ears and prevents an environment from forming in the ear canal that contributes to the growth of fungi, germs and bacteria.

2. The composition of claim 1, further including a surfactant.

3. The composition of claim 2, wherein the surfactant is chosen from at least one of propylene glycol, polysorbate, and aloe.

4. The composition of claim 1, further comprising an agent to improve viscosity.

5. The composition of claim 1, further comprising an agent to enhance solubility.

6. The composition of claim 1, wherein the antiseptic/anti-fungal agent is chosen from at least one of the following: cetylpyridinium chloride, chlorhexidine gluconate, hexetidine, sanguinanine, triclosan and benzalkonium chloride.

7. The composition of claim 1, further comprising an anti-fungal agent.

8. The composition of claim 7, wherein the anti-fungal agent comprises approximately 0.001% to 10% by weight of the composition.

9. The composition of claim 8, wherein the anti-fungal agent comprises approximately 6% by weight of the composition.

10. The composition of claim 1, further comprising a healing agent.

11. The composition of claim 10, wherein the healing agent has a zinc ion availability of 100.

12. The composition of claim 10, wherein the healing agent is chosen from at least one of: zinc acetate, zinc chloride, and mint extract oils.

13. The composition of claim 1, wherein
the carrier agent comprises approximately less than 100 percent by weight of the composition;
the antiseptic agent comprises approximately 0.001% to 10% by weight of the composition;
the buffering system comprises approximately 0.1% to 5% by weight of the composition; and
the drying agent comprises approximately 0.1% to 20% by weight of the composition.

14. The composition of claim 1, wherein
the carrier agent comprises water;
the antiseptic agent comprises more than approximately 1% by weight of the composition;
the buffering system comprises approximately 0.7% by weight of the composition; and
the drying agent comprises approximately 0.65% by weight of the composition.

15. A method for treating and/or preventing afflictions of the ear, comprising the steps of:
providing a composition, wherein the composition includes:
a carrier agent,
an anti-fungal agent present in an amount sufficient to deter fungal growth in an ear,
a buffering system, wherein the buffering system buffers the pH of the composition to an approximately neutral pH, and
a drying agent, wherein the drying agent helps prevent excess moisture in the ear and prevents an environment from forming in an ear canal that contributes to growth of fungi, germs and bacteria; and
washing the ear canal with the composition.

16. The method of claim 15, wherein said method soothes ear canals and reduces inflammation due to ear infections.

17. The method of claim 15, wherein the composition provided has a balanced viscosity range and surface dwell time, and is user friendly.

18. The method of claim 15, wherein the composition further comprises a preservative and has an enhanced shelf life.

19. The composition of claim 15, further including a surfactant.

20. The composition of claim 15, wherein the surfactant is chosen from at least one of propylene glycol, polysorbate, and aloe.

21. The composition of claim 15, further including an antiseptic agent.

22. The composition of claim 22, wherein the antiseptic agent is present in an amount greater than approximately 1% by weight.

23. The composition of claim 15, wherein the anti-fungal agent is present in an amount greater than approximately 6% by weight

24. A composition comprising:
an osmotic agent of approximately 0.1 to approximately 2 percent by weight of the composition;
a buffering agent of approximately 0.1 to approximately 5 percent by weight of the composition, wherein the buffering agent comprises at least one of dibasic sodium phosphate and monobasic sodium phosphate;
an agent to enhance solubility, of approximately 0.1 to approximately 12 percent by weight of the composition;
a surfactant of approximately 0.001 to approximately 5 percent by weight of the composition;
an antiseptic of greater than approximately 1 percent by weight of the composition;
an anti-fungal agent of greater than approximately 6 percent by weight of the composition;
a healing agent of approximately 0.001 to approximately 5 percent by weight of the composition;
a drying agent of approximately 0.1 to approximately 20 percent by weight of the composition;
an agent to enhance viscosity, of approximately 0.1 to approximately 5 percent by weight of the composition;
aloe concentrate of approximately 2 percent by weight of the composition; and
a solvent of less than approximately 100 percent by weight of the composition.
